(19) United States
(12) Patent Application Publication
(10) Pub. No.: US 2006/0246584 A1
(43) Pub. Date: Nov. 2, 2006

(54) In-vitro method for the production of a homologous stented tissue-engineered heart valve

(76) Inventor: Bruno Covelli, Suhr (CH)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 10/523,618

(22) PCT Filed: Sep. 4, 2002
(86) PCT No.: PCT/EP02/09906

§ 371 Date: Apr. 25, 2006

(30) Foreign Application Priority Data
Aug. 1, 2002 (DE) 10235237.2

(51) Int. Cl. C12N 5/08

(52) U.S. Cl. 435/396; 623/915; 435/401; 623/2.13

(57) ABSTRACT

The invention relates to an in-vitro method for the production of a homologous stented tissue-engineered heart valve.

Every year in the USA alone approx. 20,000 patients die from the consequences of a heart valve dysfunction, and more than 60,000 patients are forced to have one or more heart valves replaced surgically because of an already detected dysfunction. Possible replacements for the patient's own heart valve are either mechanical or biological valve prostheses (xenografts), and less often cryo-preserved or glutaraldehyde-fixed homografts are used.

However, mechanical valve prostheses often lead to foreign body reactions with thromboembolic complications, which are promoted by the flow conditions in the heart, which are altered by the artificial heart valve. Lifelong anticoagulation treatment is therefore necessary for the patient affected, leading to a permanently increased risk of haemorrhaging. Infections are a further, often life-threatening complication for the patient.

Xenografts are usually pig valves treated with glutaraldehyde. Pig valve prostheses can be employed with good results in older patients, but tend to degenerate after only approx. 12 to 15 years, so that as a rule they are unsuitable for young people. There is furthermore an increased risk of infection with pig valve prostheses compared with the healthy heart. Moreover, pig valves tend to calcify, and for this reason are unsuitable for use in children and young people, who have an increased calcium metabolism. Finally, they are likewise exogenous tissue, which with a certain probability is recognized as foreign by the endogenous immune system and can thus trigger adverse immune processes

Homografts, i.e. fixed heart valves isolated from human donors, are available as a third possibility. Homografts are indeed relatively resistant to infections, but are likewise exogenous tissue which with a certain probability causes immune reactions. Moreover, homografts, just like pig valve prostheses, tend to calcify and are therefore subject to considerable degeneration, which as a rule necessitates re-operation after 7 to 12 years. The availability of homografts moreover is only extremely limited.

In addition to the disadvantages already described for valve prostheses used hitherto as a replacement valve, i.e. triggering of immune reactions, increased risk of infection, risk of thromboembolic processes and tendency to degenerate, all the valves known hitherto have the common feature that they are made of inorganic material or fixed organic material and they therefore lack important properties of a living matrix, e.g. the capacity for repair processes, for reconfiguration or for growth. It follows from this, inter alia, that for child valve patients re-operation hitherto regularly had to be accepted. In addition to the inherent risk of any heart operation, however, the morbidity and mortality risk increases with every re-operation, since considerable fusions occur in the thorax due to the preceding operations.

There is therefore an urgent need for a replacement heart valve which avoids the disadvantages described above. For this purpose, production of artificial heart valves by tissue engineering has already been proposed. Tissue engineering is concerned with the development of “biohybrid” implants which grow on to tissue or even on to entire organ systems in the body. The production of biohybrid heart valves in the form of individual valve leaflets has also already been described; however, the heart valve leaflets produced by tissue engineering hitherto had the disadvantage that they have inadequate, insufficient connective tissue structures and therefore could not withstand the flow conditions prevailing in the hear after the biodegradable support structure had dissolved.

DE 19919625 describes an in vitro method for the production of a homologous heart valve. The heart valve described there is built up on a biodegradable support, which is incubated with homologous fibroblasts and/or myofibroblasts to form a connective tissue-like matrix and is then colonized with endothelial cells. The connective tissue-like matrix is then transferred into a bioreactor for maturing of the tissue. This heart valve is best adapted to the flow conditions in the human body. At the time of its implantation, the heart valve described in DE 19919625 almost entirely comprises autologous cell material, which is then sewn into the receiving heart Under certain circumstances, one disadvantage of this heart valve could be that the surgical implantation is technically difficult to perform. There could moreover be a problem if the suture has to pass through the autologous, tissue-engineered tissue to be sewn in. Because of the extremely high load the heart valve is subsequently exposed to in the human body, tears could occur in the region of the suture.

The object of the invention is therefore to provide improved homologous heart valves and a method for their production.

According to the invention, the object is achieved by an in vitro method for the production of a homologous heart valves which comprises the following steps:
provision of a biodegradable support (scaffold),
colonization of the support with homologous fibroblasts and/or myofibroblasts to form a connective tissue matrix,
optionally colonization of the connective tissue matrix with endothelial cells
fixing of the connective tissue matrix to a non-degradable or poorly degradable frame construction (stent),
   wherein, before or after the fixing to the frame construction, the connective tissue matrix optionally colonized with endothelial cells is introduced into a pulsatile flow chamber in which it can be exposed to increasing flow rates, and the flow rate is increased continuously or discontinuously.

In an alternative method, a homologous heart valve is produced by
provision of a biodegradable support (scaffold) which is firmly connected to a non-degradable frame construction (stent),
colonization of the support with homologous fibroblasts and/or myofibroblasts to form a connective tissue matrix,
optionally colonization of the connective tissue matrix with endothelial cells,
introduction of the frame construction with the connective tissue matrix connected thereto into a pulsatile flow chamber in which it can be exposed to increasing flow rates,
continuous or discontinuous increasing of the flow rate.

Homologous heart valves which have all the advantages of the heart valve known from DE19919625 and moreover avoid a suture having to be passed through the connective tissue structures of the heart valve at the time of implantation of the valve can be produced by the methods according to the invention. They withstand the flow conditions prevailing in the body and are easy to implant surgically.

The methods for the production of the heart valve according to the invention and the heart valve produced by them are to be explained in more detail in the following.

In the following description, the term “support” means an acellular structure which, as explained in more detail below, is formed from either synthetic fibres or an acellular connective tissue framework. The term “matrix” designates a connective tissue structure which contains, in addition to fibroblasts and myofibroblasts, typical constituents of an extracellular matrix, namely collagen, elastin and glycosaminoglycans. Structures called a matrix typically contain support constituents undergoing degradation or no longer contain any support constituents.

For carrying out the method according to the invention, a biodegradable support is first provided. The support material on the one hand should be stable in this context for a certain period of time in order to allow adequate colonization or penetration with fibroblasts and/or myofibroblasts and to be able to achieve the formation of a connective tissue matrix, and on the other hand should be able to be dissolved in total within an acceptable time, which ideally is shorter than the time taken for the formation of the homologous valve prosthesis. It is preferable for the degradation to start after approx. 8 days; as a rule, it should be concluded in less than 3 months, preferably already after 4 to 6 weeks.

After formation of a solid connective tissue matrix structure, the degradable support of which does not yet have to be dissolved, this is optionally colonized with endothelial cells. After the colonization has taken place, the connective tissue matrix is applied to a non-degradable or poorly degradable frame construction. Alternatively, however, a support already firmly connected to a frame construction can be subjected to the colonization steps. The possible alternative variants of the method are to be described in more detail in the following.

In one variant of the method according to the invention, the biodegradable support (scaffold) is first colonized with homologous fibroblasts and/or myofibroblasts to form a connective tissue matrix. The matrix is then optionally colonized with endothelial cells. According to the invention, the preformed structure analogous to a heart valve can now be introduced, in a further method step for maturing the tissue and optimizing the haemodynamic function, into a pulsatile flow chamber in which it can be exposed to increasing flow rates. By continuous or discontinuous increasing of the flow rate, it is adapted here to the flow conditions in the human body. For further stabilization, the structure analogous to a heart valve is fixed to a biocompatible frame construction of non-degradable or poorly degradable material, which is optionally introduced again into the pulsatile flow chamber. In the case where adaptation to the flow conditions in the human heart is carried out in the flow chamber after fixing to the frame construction, the first incubation in the pulsatile flow chamber can be omitted. Vital heart valve prostheses which withstand the flow conditions in the human body are obtained by these methods.

In an alternative variant of the method according to the invention, the biodegradable support (scaffold) can already be firmly connected to the non-degradable or poorly degradable frame construction (stent) before the colonization. In a further step, the support connected to the frame construction is then colonized with homologous fibroblasts and/or myofibroblasts and then optionally with endothelial cells to form a connective tissue matrix. For maturing the tissue and optimizing the haemodynamic function, the preformed structure analogous to a heart valve is then introduced into a pulsatile flow chamber, in which it can be exposed to increasing flow rates. By continuous or discontinuous increasing of the flow rate, a vital heart valve prosthesis which withstands the flow conditions in the human body is likewise obtained by this procedure.

Overall, the following method variants thus result:

Variant 1:

provision of a support without a frame construction
colonization
adaptation in a pulsatile flow chamber
fixing to a non-degradable or poorly degradable frame construction (stent)
if appropriate re-adaptation of the “stented” heart valve
   Variant 2:
provision of a support without a frame construction
colonization
fixing to a non-degradable or poorly degradable frame construction (stent)
adaptation of the “stented” heart valve
   Variant 3:
provision of a support on a non-degradable or poorly degradable frame construction
colonization
adaptation

The support material is preferably a structure built up from polymer fibres around a porous polymer structure or an acellular biological tissue. Suitable synthetic polymers for this use also include bioerodable polymers, such as e.g. polyglycolic acid (PGA), polylactic acid, (PLA), polyhydroxyalkanoate (PHA) and poly-4-hydroxybutyrate (P4HB), polycaprolactones, (PLGA), polycarbonates, polyamides, polyanhydrides, polyamino acids, polyorthoesters, polyacetates, polycyanoacrylates and degradable polyurethanes, and non-erodable polymers, such as polyacrylates, ethylene/vinyl acetate polymers and other substituted cellulose acetates as well as derivatives thereof. Polyesters are preferred here.

Preferred biodegradable polymers include polymers chosen from the following group: polyesters of hydroxycarboxy acids, polyanhydrides of dicarboxy esters and copolymer of hydroxycarboxy acids and dicarboxy esters.

In a further embodiment, the material is made of a synthetic polymer of at least one of the following monomers: glycolide, lactide, p-dioxanone, caprolactone, trimethylene carbonate and butyrolactone. In particular embodiments, the material is chosen from a group consisting of polymers or copolymers of glycolic acid, lactic acid and sebacic acid. Polyglycolic acid polymers are preferred here.

These polymers can be used either in the pure form or in mixtures of two or more of the substances mentioned or mixtures of these substances with further biodegradable polymers. In a preferred embodiment, a copolymer of 85% PGA and 15% PLA is used.

In a further preferred embodiment, the support is produced from a polyhydroxyalkanoate (PHA). The PHA in this context can be coated with a further non-degradable polymer. A preferred polyhydroxyalkanoate for this use degrades in vivo within less than 9 months, even more preferably in less than 6 months and most preferably in less than 3 months. A preferred composition of the polyhydroxyalkanoates comprises 2-, 3-, 4- or 5-hydroxy acids, e.g. poly-4-hydroxybutyrates. The composition can furthermore comprise a poly-4-hydroxybutyrate-co-3-hydroxybutyrate and combinations thereof. Poly-4-hydroxybutyrate is most preferred in this context.

In a further particular embodiment, the support is made of homopolymers and copolymers with any desired combination of the following monomers: 3-hydroxybutyrates, 3-hydroxyvalerate, 3-hydroxypropionate, 2-hydroxybutyrate, 4-hydroxybutyrate, 4-hydroxyvalerate, 3-hydroxyhexanoate, 3-hydroxyheptanoate, 3-hydroxyoctanoate, 3-hydroxynonanoate, 3-hydroxytridecanoate, 3-hydroxytetradecanoate, 3-hydroxypentadecanoate, 3-hydroxyhexadecanoate, 3-hydroxyheptadecanoate and 3-hydroxyoctadecanoate.

It has proved appropriate to use biodegradable supports having a polymer density of approx. 40 to 120 mg/cm³. Below 40 mg/cm³ the polymer fabric is too unstable, and above 120 mg/cm³ the fabric is too dense to allow penetration of fibroblasts within an acceptable period of time. In preferred embodiments, the density of the biodegradable support is 50 to 80 mg/cm³, particularly preferably 70 mg/cm³. In the present invention, a polymeric support from Albany International Research, Mensville, Mass., USA having a density of approx. 70 mg/cm³ was used with good results, as wells as a polymeric support from TRANSOME INC., Palm Bay, Fla. USA.

The fibres of the support can have a diameter of 6 to 20 μm, preferably 10 to 18 μm. However, fabrics having other fibre thicknesses are also conceivable, but on the one hand these must impart a certain stability to the support, and on the other hand they must allow colonization and penetration of the support with fibroblasts or myofibroblasts. Pore sizes of 80-240 μm have proved favourable for porous (sponge-like) polymer forms. The pores can be achieved by the so-called salt leaching technique, which is known to the expert.

Instead of a synthetic support, as described above, the use of an acellular connective tissue framework is conceivable. Thus, for example, a pig valve could be converted into an immunologically neutral tissue (Bader et al., Eur. J. Cardiothorac. Surg. 14, 279, 1998), which could then be colonized with homologous cells. Human heart valves can also be colonized again after neutralization.

The biodegradable support is first incubated with a fibroblast population. If homologous fibroblasts and/or myofibroblasts, i.e. fibroblasts and/or myofibroblasts from a human, but not necessarily the patient, are used, it should be ensured that the HLA types are the same.

Fibroblast populations can be obtained in this context e.g. from peripheral blood vessels, both arteries and veins. The arteria radialis of the forearm which, because of the double arterial supply of the arm, in most cases is available for harmless explantation, is particularly suitable for this. Alterntively, vessel cells can be obtained from blood vessels of the leg, e.g. the vena saphena. The myofibroblasts and endothelial cells can furthermore be obtained from bone marrow precursor cells or from pluripotent stem cells or genetically manipulated cells.

The cells can be obtained, for example, from vessel fragments by a procedure in which, as described in Zünd et al. (Eur. J. Cardiothorac. Surg. 13, 160, 1998), the pieces of tissue are first cut into tissue fragments and are incubated for approx. 1 to 2 weeks under normal cell culture conditions (37° C., 5% CO₂, 95% atmospheric humidity) until the cells form a confluent cell layer on the base of the culture dish. They are then subjected to several passages in order to obtain a cell culture which is free from residual tissue material. After two to three passages, the mixed cell populations can be purified by a procedure in which they are incubated with a fluorescence marker specific for endothelial cells (Dil-Ac-LDL, from Medical Technologies Inc., Stoughton, Mass.) and are separated by means of flow cytometry (FACStar Plus, Becton Dickinson). Cells marked with fluorescence are endothelial cells, non-marked cells are fibroblasts and myofibroblasts. These are cultured for a further two to three weeks and subjected to two to four passages during this period of time in order to obtain a sufficient number of cells for subsequent colonization of the support.

A fibroblast/myofibroblast culture purified as described or any other pure fibroblast/myofibroblast culture can now be employed for colonization of the polymer support. For this, approx. 10⁵ to 6×10⁸ fibroblasts and/or myofibroblasts are employed per square centimetre of surface of the support. “Surface” in this case does not mean the actual surface of the polymer, but the areas detectable in a plane when the support is viewed from above. The fibroblasts are conventionally given a time of 60 to 90 min to adhere to the support. The supernatant medium can then be removed and a fibroblast suspension added again. Ideally, however, 2 to 36 hours, preferably 24 hours, are allowed to elapse between the first and second addition of fibroblast suspension.

In a preferred embodiment of the method according to the invention, fibroblasts and/or myofibroblasts are added a further 3 to 14 times, particularly preferably 5 to 10 times, to the support or the matrix which gradually forms after the first addition of fibroblasts.

Under the conditions conventionally used for cell growth of fibroblasts (e.g. 5% CO₂, incubation at 37° C., sterile medium), a solid connective tissue structure develops after approx. one to three weeks. In a preferred embodiment, this structure is then incubated with a pure endothelial cell suspension. The endothelial cells, in the same way as the fibroblasts, can be concentrated by FACS and then expanded in several passages (preferably 3). For endothelial cells it is also preferable to repeat the colonization several times, e.g. 3 to 14 times, with in each case approx. 10⁵ to 5×10⁸ endothelial cells. In preferred embodiments, the colonization with endothelial cells is repeated 5 to 10 times. There should be at least 60 min, but preferably 2 to 24 hours, between two colonization steps. However, the endothelial cell colonization step is optional.

The cells used to colonize the support are preferably human cells. However, it is particularly preferable to used autologous fibroblasts and/or myofibroblasts and optionally endothelial cells. For this, tissue is removed from the patient, e.g. from one of his vessels, in whom a heart valve is to be replaced. As already mentioned above, the arteria radialis and the vena saphena or bone marrow are suitable for this. The use of autologous cells for construction of the heart valve has the substantial advantage that after implantation into the patient, the valve is not exogenous tissue and immune reactions against the artificial heart valve appear to be as good as ruled out.

Approx. 14 days after the optional addition of the endothelial cells, a tissue having a superficial single cell layer of endothelial cells and a connective tissue base structure can be detected histologically and immunohistochemically.

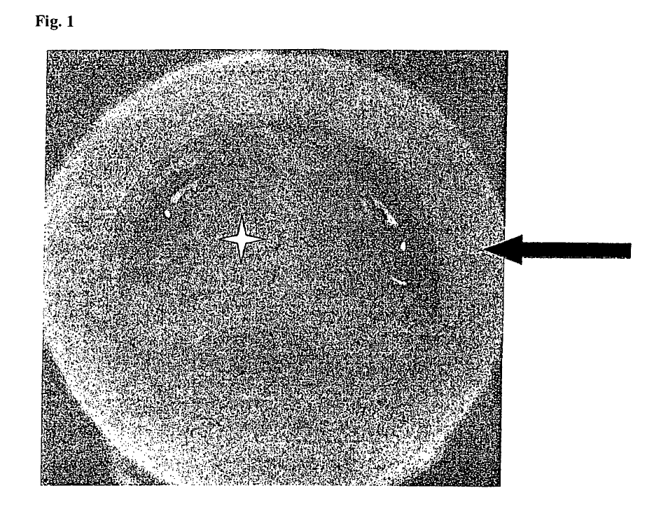

In a preferred embodiment, the connective tissue matrix has the form of a heart valve and is provided with a broad connective tissue edge, the so-called suture ring, which is fixed on to a circular frame construction. An example of such a heart valve with a suture ring is shown in FIG. 1.

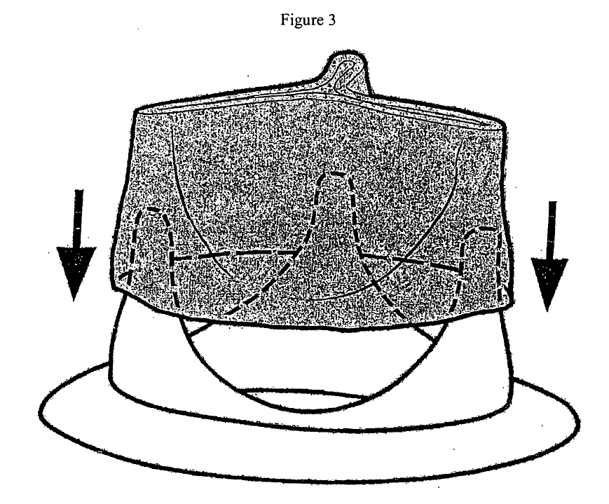

In a further preferred embodiment, the connective tissue matrix has the form of a tape or a ring. This embodiment requires a suture ring, which is provided with triple-peaked support structure, as the frame construction. The tape or ring is then passed around this triple-peaked structure. An example of this embodiment is shown in FIG. 3. The diameter of the frame construction can be chosen individually in this context and depends on the anatomical requirements of the patient.

The frame construction (stent) according to the invention can be constructed from various materials. In order to impart to the newly produced tissue the longest possible life and strength, the material should be constructed from non-degradable biocompatible material or, alternatively, from poorly degradable biocompatible material, e.g. carbon, PTFE, Dacron, metal or PHA, preferably poly-3-hydroxybutyrate (P3HB). “Poorly degradable” in this context means a degradation duration of more than one year.

The connective tissue matrix can be fixed to the frame construction by conventional suturing. In one embodiment, the matrix can be fixed to the frame construction by means of fibrin adhesive. The connective tissue matrix is particularly preferably fixed to the support by conventional suturing in combination with fibrin adhesive. The shape of the individual heart valve leaflets can likewise be stabilized either by a suture or by gluing with fibrin adhesive, by a procedure in which the edges encircling the peaks in the direction of the centre of the circle are sewn or glued.

According to the invention, in a further method step the preformed structure analogous to a heart valve can now be introduced into a pulsatile flow chamber in which it can be exposed to increasing flow rates. It has been found that the formation of a connective tissue matrix which is resistant to flow can be achieved by slow adaptation of the flow rates.

The bioreactor described in DE19919625, for example, is suitable for carrying out the method according to the invention.

In one embodiment of the invention, flow rates of between 5 ml/min and 8,000 ml/min, preferably between 30 ml/min and 5,000 ml/min, particularly preferably 50 ml/min to 2,000 ml/min are used. The data relate to the flow through the valve prosthesis. Flow rates of 50 to 100 ml/min have proved suitable as the initial flow rate. The heart valve is charged with these flow rates e.g. with a pulse frequency of 5 to 10 pulses per minute. The flow rate is then increased continuously or discontinuously to up to 5,000 ml/min. At the same time, the pulse frequency is raised to up to 180 pulses/min. The data stated are the limit values, which normally are not exceeded.

In preferred embodiments, the flow rate is increased up to 2,000 ml/min, while the pulse frequency is raised to 70 to 100, preferably 80 pulses/min. The load on the stabilizing heart valve is thus adapted virtually to physiological conditions. It has proved favourable, but not necessary, to increase the flow rate and the pulse frequency after every approx. 24 to 48 hours. Thus, for example, starting from a flow rate of 50 to 100 ml/min and a pulse rate of 5 to 10 pulses/min on day 1 of the duration of stay in the pulsatile flow chamber, an increase to 300 ml/min at 20 to 25 pulses/min can be envisaged on day 3, to 700 ml/min and 35 to 45 pulses/min on day 5, to 1,000 ml/min and 50 to 60 pulses/min on day 7, to 1,300 ml/min and 70 to 80 pulses/min on day 9, to 1,500 ml/min and approx. 100 pulses/min on day 11, to 1,750 ml/min and approx. 120 pulses/min on day 13 and to 2,000 ml/min and 140 pulses/min on day 15. However, a very much slower increase in the flow rates and pulse frequency or an increase to higher flow rates and pulse frequencies may be appropriate, depending on the time available, the size of the valve, the size and age of the patient etc.

In one embodiment of the invention, the systemic pressures prevailing in the pulsatile flow chamber are adjusted to 10 to 240 mm Hg. Systemic pressures of 60 to 140 are preferred, and systemic pressures of 80 to 120 mm Hg are particularly preferred.

The homologous or autologous heart valve produced by means of the method according to the invention has substantial advantages compared with conventional mechanical and biological heart valves. Thus, in its preferred embodiment, the heart valve according to the invention comprises autologous tissue, i.e. tissue of the patient scheduled for the heart valve operation, and a biocompatible material which stabilizes it further, which is used as the frame construction. A foreign body reaction of the valve recipient to the implant is thereby avoided. The risk of infection to recipients of a heart valve according to the invention is thus reduced considerably. An anticoagulation therapy is not necessary; the risk of haemorrhagic complications is therefore eliminated. By far the most convincing advantage of the heart valve according to the invention, however, is the fact that it is living tissue and is therefore capable of permanent regeneration and repair after implantation. In its preferred embodiment, the heart valve according to the invention furthermore combines the advantages of a completely autologous heart valve prosthesis and the very good haemodynamic functions of synthetic heart valve prostheses. In the end, with the heart valves according to the invention significantly fewer degenerative changes and/or dysfunctions are to be expected by using the biocompatible frame construction, even during relatively long use, which significantly increases the life of the heart valve and therefore significantly reduces the risk of re-operation.

The heart valve according to the invention comprises a connective tissue inner structure which contains, in addition to fibroblasts and myofibroblasts, substantial constituents of a normal extracellular matrix, namely collagen, elastin and glycosaminoglycans. The valves according to the invention thus have a content of collagen (26-60%), elastin (2-15%) and glycosaminoglycans corresponding to the native valve or the native valve leaflet. This connective tissue inner structure built up on a biodegradable support (scaffold) and colonized with endothelial cells is stabilized further by a biocompatible frame construction. The connective tissue structure is fixed to the biocompatible frame construction as described above. Such a heart valve prosthesis combines the advantages of an autologous heart valve prosthesis and the very good surgical implantability and function of synthetic heart valve prostheses.

It was possible to demonstrate that the heart valves according to the invention withstand flow rates of more than 2,000 ml/min, corresponding to the flow conditions prevailing in an adult human heart. An autologous heart valve which is unconditionally suitable for implantation into child and also adult patients can thus be provided according to the invention.

The following figures and examples explain the invention.

FIG. 1 shows a support (see star) preformed from a polymer and having a suture ring (see arrow), after colonization with fibroblasts/myofibroblasts and endothelial cells.

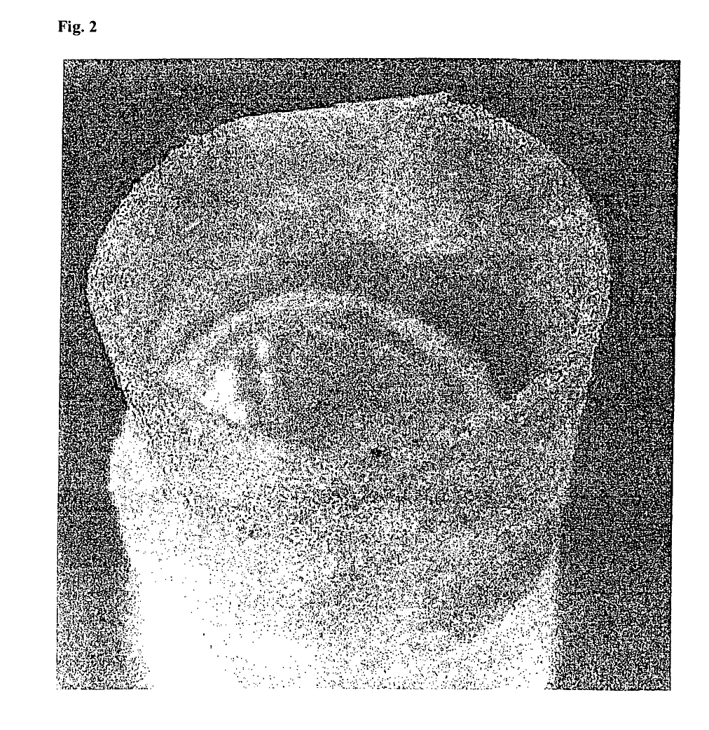

FIG. 2 show a tubular colonized matrix, from which rings of 3.5 cm width, which can be laid around the frame construction shown in FIG. 3, can be cut.

FIG. 3 shows a diagram of the frame construction from FIG. 3 with the colonized matrix passed around the triple-peaked support structure.

### EXAMPLE 1

### Production of Valve-Carrying Conduit (Tube) Supports

A non-woven polyglycolic acid polymer (fibre diameter: 12-15 p. m, polymer density: 70 mg/ml, Albany International Research, Mansfield Mass., USA) is used to produce the valve-carrying conduit support. The polymer is cut such that it forms tubes of 19 mm diameter. 3 triangular leaflets are inserted into this conduit. This support can be used for production of 3-leaflet valves, i.e. pulmonary, aortic and tricuspid valves. For mitral valves, 2 leaflets are inserted.

### EXAMPLE 2

### Production of a Three-Leaflet Heart Valve Prosthesis Stabilized by a Frame Construction

A three-leaflet valve-carrying conduit support is sterilized and laid in medium (DM EM, GIBCO BRL-Life Technologies) for 24 hours in order to steep the polymer surface.

Thereafter, the valve-shaped support is colonized with 4 million fibroblasts per square centimetre of surface every 90 minutes 6 times in total. The colonized support is furthermore incubated for 2 weeks (5% CO₂, 37° C., 95% atmospheric humidity). The medium is changed under sterile conditions every 4 days. Endothelial cells are then applied to the colonized valve-shaped support (3-4 million endothelial cells per square centimetre of surface, 6 colonizations every 90 minutes). After a further 2 weeks, the tissue formed is turned inside out over a prepared biocompatible frame construction and firmly connected to the frame construction by a suture. The entire construction is then introduced into the flow chamber of the bioreactor under sterile conditions and installed here in the flow-through position. The bioreactor is now filled with medium and placed in the cell incubator. After the connection to the pump outside the incubator has been established via the compressed air hose, minimal pulsatile flows (50 ml/min) are started. The flow rate and pulse rate are increased in 2-day steps to 100 ml/min (pulse 10), 300 ml (pulse 25), 700 ml (pulse 35) and 1,000 ml (pulse 60), for a further 4 days in total. The tissue now formed is subsequently (after 14 days) removed under sterile conditions and reserved for biochemical, histological and mechanical analysis.

### EXAMPLE 3

### Production of Three-Leaflet Heart Valve-Shaped Supports Stabilized by a Multi-Peaked Frame Construction

1-2 mm thick non-woven copolymer of polyglycolic acid (PGA) and polyhydroxyalkanoate (PHA) (fibre diameter 12-15 μm, polymer density 70 mg/ml) is used to produce the heart valve-shaped support and is cut such that a tape 3.5 cm wide and 8.0 cm long is formed. This tape is connected at the end points using absorbable suture material and additionally welded at the overlapping zones with application of heat (60-70° C.). The ring now formed is turned inside out over a triple-peaked frame construction (Dacron) and is connected to this by means of a suture and using fibrin adhesive. The individual heart valve leaflets were subsequently shaped into the bulging three-leaflet form typical of heart valves over the frame construction, again with application of heat.

### EXAMPLE 4

### Production of a Three-Leaflet Heart Valve Prosthesis Stabilized by a Triple-Peaked Frame Construction

A three-leaflet support stabilized by a triple-peaked frame construction is sterilized and laid in medium (DM EM, GIBCO BRL-Life Technologies) for 24 hours in order to steep the polymer surface. Thereafter, the valve-shaped support is colonized with 4 million fibroblasts per square centimetre of surface every 90 minutes 6 times in total. The colonized support is furthermore incubated for 2 weeks (5% CO₂, 37° C., 95% atmospheric humidity). The medium is changed under sterile conditions every 4 days. Endothelial cells are then applied to the colonized valve-shaped support (3-4 million endothelial cells per square centimetre of surface, & colonizations every 90 minutes). The entire construction is then introduced into the flow chamber of the bioreactor under sterile conditions and installed here in the flow-through position. The bioreactor is now filled with medium and placed in the cell incubator. After the connection to the pump outside the incubator has been established via the compressed air hose, minimal pulsatile flows (50 ml/min) are started. The flow rate and pulse rate are increased in 2-day steps to 100 ml/min (pulse 10), 300 ml (pulse 25), 700 ml (pulse 35) and 1,000 ml (pulse 60), for a further 4 days in total. The tissue now formed is subsequently (after 14 days) removed under sterile conditions and reserved for biochemical, histological and mechanical analysis.

1. An in vitro method for the production of a homologous heart valve, comprising the steps of:
a) providing a biodegradable support,
b) colonizing the support with homologous fibroblasts or myofibroblasts cells or a combination thereof to form a connective tissue matrix,
c) optionally colonizing the connective tissue matrix with endothelial cells, and
d) fixing the connective tissue matrix to a non-degradable or poorly degradable frame construction,
wherein, before or after the fixing of the frame construction, the connective tissue matrix optionally colonized with endothelial cells is introduced into a pulsatile flow chamber in which it can be exposed to increasing flow rates, and the flow rate is increased continuously or discontinuously.

2. An in vitro method for the production of a homologous heart valve, comprising the following steps:
a) providing a biodegradable support which is firmly connected to a non-degradable or poorly degradable frame construction
b) colonizing the support with homologous fibroblast or myofibroblasts cells or a combination thereof to form a connective tissue matrix,
c) optionally colonizing the connective tissue matrix with endothelial cells,
d) introducing the frame construction with the connective tissue matrix connected thereto into a pulsatile flow chamber in which it can be exposed to increasing flow rates, and
e) continuously or discontinuously increasing of the flow rate.

3. The method according to claims 1 or 2, wherein the biodegradable support comprises a biodegradable polymer matrix or an acellular biological matrix.

4. The method of claim 3, wherein the support comprises a polyglycolic acid (PGA), polylactic acid (PLA), polyhydroxyalkanoate (PHA), poly-4-hydroxybutyrate (P4HB) or a mixture of two or more of these polymers.

5. The method according to claims 1 or 2, wherein the support has a polymer density of 40 to 120 mg/cm³.

6. The method according to claims 1 or 2, wherein the support comprises a porous polymer having a pore size of 80 to 240 μm.

7. The method according to claims 1 or 2, wherein the fibers of the support have a diameter of 6 to 20 μm.

8. The method of claim 3, wherein the support comprises an acellular connective tissue framework of an animal or human heart valve.

9. The method according to claims 1 or 2, wherein the step of colonization with fibroblast or myofibroblast cells or a combination thereof repeated 3 to 14 times.

10. The method according to claims 1 or 2, wherein approximately 10⁵ to 6×10⁸ fibroblast or myofibroblasts cells or a combination thereof are employed per square centimeter of support.

11. The method according to claims 1 or 2, wherein the step of colonization with endothelial cells is repeated 3 to 14 times.

12. The method according to claims 1 or 2, wherein approximately 10⁵ to 5×10⁸ endothelial cells are employed per square centimeter of support.

13. The method according to claims 1 or 2, wherein the cells are human cells.

14. The method according to claims 1 or 2, wherein the cells are autologous cells.

15. The method according to claims 1 or 2, wherein the frame construction comprises a biocompatible material.

16. (canceled)

17. The method according to claims 1 or 2, wherein the support is fixed to the frame construction by means of conventional suturing, fibrin adhesive, or a combination thereof.

18. The method according to claim 1 or 2, wherein flow rates of 5 ml/min to 8,000 ml/min are established in the pulsatile flow chamber.

19. The method according to claims 1 or 2, wherein the flow rate is increased over a period of 1 week to 12 weeks.

20. The method according to claims 1 or 2, wherein the initial flow rate is 50 to 100 ml/min.

21. The method according to claims 1 or 2, wherein the initial pulse frequency is 5 to 10 pulses/min.

22. The method according to claims 1 or 2, wherein the flow rate is increased to 5,000 ml/min.

23. The method according to claims 1 or 2, wherein the pulse frequency is increased to 180 pulses/min.

24. The method according to claims 1 or 2, wherein systemic pressures of 10 to 240 mm Hg are established in the pulsatile flow chamber.

25. An autologous heart valve that has been produced by the method according to claims 1 or 2.

26. An autologous heart valve having a connective tissue inner structure surrounded by an endothelial cell layer, wherein the connective tissue inner structure is fixed to a non-degradable or slowly degradable frame constructions.

27. The autologous heart valve according to claim 26, wherein a collagen density of 20 to 60% exists in the connective tissue inner structure.

28. The autologous heart valve according to claim 27, wherein the heart valve withstands the flow conditions in the human heart.
